(19) United States
(12) Patent Application Publication
(10) Pub. No.: US 2023/0389809 A1
(43) Pub. Date: Dec. 7, 2023

(54) METHOD AND APPARATUS FOR MEASURING BLOOD PRESSURE USING RADAR

(71) Applicant: ELECTRONICS AND TELECOMMUNICATIONS RESEARCH INSTITUTE, Daejeon (KR)

(72) Inventors: Sung Jin YOO, Daejeon (KR); Hyung Ju KIM, Daejeon (KR); Byung Jang JEONG, Daejeon (KR)

(73) Assignee: ELECTRONICS AND TELECOMMUNICATIONS RESEARCH INSTITUTE, Daejeon (KR)

(21) Appl. No.: 18/083,321

(22) Filed: Dec. 16, 2022

(30) Foreign Application Priority Data
Jun. 7, 2022 (KR) 10-2022-0069030

(51) Int. Cl. A61B 5/021; A61B 5/0205; A61B 5/024

(52) U.S. Cl. CPC A61B 5/02108; A61B 5/02427; A61B 5/0205

(57) ABSTRACT

A blood pressure measurement method using at least one radar in a blood pressure measurement apparatus may comprise: measuring a radar signal for at least one body part; extracting a heartbeat signal from the radar signal; selecting one or more peak signals from the heartbeat signal; calculating an area under a curve (AUC) of each of the selected one or more peak signals; and calculating blood pressure from the calculated AUC on the basis of a derived relational expression between AUC and blood pressure.

### CROSS-REFERENCE TO RELATED APPLICATIONS

This application claims priority to Korean Patent Application No. 10-2022-0069030, filed on Jun. 7, 2022, with the Korean Intellectual Property Office (KIPO), the entire contents of which are hereby incorporated by reference.

### BACKGROUND

### 1. Technical Field

Example embodiments of the present disclosure relate to a technique for detecting a biosignal using a radar, and more specifically, to a method and apparatus for measuring blood pressure using a radar.

### 2. Related Art

Traditionally, a cuff is used to measure blood pressure. Blood pressure is measured by detecting the sound generated by a difference in blood pressure between the cuff and blood vessels. In this way, although blood pressure can be measured relatively accurately with a simple principle, it takes a long time to measure blood pressure and gives people discomfort when pressure is applied. In addition, it is difficult to consecutively obtain blood pressure information.

In order to compensate for the above disadvantages, recently, a method of measuring blood pressure using an electrocardiogram (ECG) or a photoplethysmogram (PPG) has been proposed. In this case, instead of directly measuring blood pressure, the blood pressure is indirectly inferred using a correlation between a pulse transit time (PTT) and blood pressure. Alternatively, blood pressure is measured using information according to a pulse shape such as an area under the curve (AUC).

The method of measuring the blood pressure using the ECG or PPG is easier to consecutively measure the blood pressure than the method of measuring the blood pressure using the cuff, but still has a limitation in that it is a method performed in a contact manner.

### SUMMARY

Accordingly, example embodiments of the present disclosure are provided to substantially obviate one or more problems due to limitations and disadvantages of the related art.

Example embodiments of the present disclosure provide a method and apparatus for measuring blood pressure using a radar capable of measuring blood pressure in a non-contact manner.

According to a first exemplary embodiment of the present disclosure, a blood pressure measurement method using at least one radar in a blood pressure measurement apparatus may comprise: measuring a radar signal for at least one body part; extracting a heartbeat signal from the radar signal; selecting one or more peak signals from the heartbeat signal; calculating an area under a curve (AUC) of each of the selected one or more peak signals; and calculating blood pressure from the calculated AUC on the basis of a derived relational expression between AUC and blood pressure.

The calculating of the AUC may include calculating an average value of the calculated AUCs from the selected one or more peak signals.

The calculating of the AUC may include normalizing a magnitude of the selected peak signal before the AUC is calculated.

The extracting of the heartbeat signal may include removing a signal related to breathing information and a noise signal from the radar signal.

According to a second exemplary embodiment of the present disclosure, a blood pressure measurement apparatus using at least one radar may comprise: a heartbeat signal extraction unit configured to extract a heartbeat signal from a radar signal reflected by at least one body part through the at least one radar; and a blood pressure measurement unit configured to calculate a parameter to be used for measuring blood pressure from the heartbeat signal and calculate the blood pressure from the parameter, wherein the blood pressure measurement unit selects one or more peak signals from the heartbeat signal, calculates an area under curve (AUC) of each of the selected one or more peak signals, and calculates the blood pressure using the calculated AUC corresponding to the parameter.

The blood pressure measurement unit may calculate the blood pressure from the calculated AUC on the basis of a derived relational expression between AUC and blood pressure.

The blood pressure measurement unit may extract amplitude information of the heartbeat signal and calculate the parameter by combining the calculated AUC and the extracted amplitude information.

The blood pressure measurement unit may extract phase information of a frequency component from a radar signal reflected by each of two body parts, calculate a pulse transit time (PTT) using the extracted phase information, and calculate the parameter by combining the calculated AUC and the calculated PTT.

The heartbeat signal extraction unit may remove a signal related to breathing information and a noise signal from the radar signal.

The blood pressure measurement unit may extract amplitude information of the heartbeat signal, calculate a PTT from a radar signal reflected by each of two body parts, and calculate the parameter by combining the calculated AUC, the extracted amplitude information, and the calculated PTT.

According to the embodiments of the present disclosure, since blood pressure can be measured in a non-contact manner, it is possible to reduce the discomfort of a subject to be measured and it can be easier to consecutively measure blood pressure than other methods.

### BRIEF DESCRIPTION OF DRAWINGS

FIG. 1 is a schematic diagram illustrating a method of measuring blood pressure using a synchronized radar according to an embodiment.

FIGS. 2 and 3 are diagrams each illustrating an example of a configuration of a board of the synchronized radar.

FIG. 4 is a schematic diagram illustrating a method of measuring blood pressure using two unsynchronized radars.

FIG. 5 is a set of diagrams showing examples of received signals of two unsynchronized radars.

FIG. 6 is a set of diagrams showing examples of received signals of two synchronized radars.

FIG. 7 is a diagram showing examples of radar signals in a synchronized time domain.

FIG. 8 is a set of diagrams showing examples of radar signals in a synchronized frequency domain.

FIG. 9 is a diagram illustrating a process of deriving a relational expression between PTT and blood pressure.

FIG. 10 is a diagram showing an example of the relational expression between PTT and blood pressure derived by the method illustrated in FIG. 9.

FIG. 11 is a diagram showing examples of amplitude information of radar signals measured at a heart part and a wrist part.

FIG. 12 is a diagram showing a relationship between the amplitude of a radar signal and blood pressure.

FIG. 13 is a diagram illustrating a process of deriving a relational expression between information about the amplitude of one radar signal and blood pressure.

FIG. 14 is a diagram showing a relationship between a shape of a radar signal and blood pressure.

FIG. 15 is a flowchart illustrating a method of deriving a relational expression between AUC measured from a radar signal and blood pressure.

FIG. 16 is a diagram showing an example of a heartbeat signal extracted from a radar signal.

FIG. 17 is a flowchart illustrating a method of measuring blood pressure using an AUC measured from a radar signal according to an embodiment.

FIG. 18 is a diagram illustrating a blood pressure measurement apparatus using a radar according to an embodiment.

FIG. 19 is a diagram illustrating a blood pressure measurement apparatus using a radar according to another embodiment.

### DETAILED DESCRIPTION OF THE EMBODIMENTS

Exemplary embodiments of the present disclosure are disclosed herein. However, specific structural and functional details disclosed herein are merely representative for purposes of describing exemplary embodiments of the present disclosure. Thus, exemplary embodiments of the present disclosure may be embodied in many alternate forms and should not be construed as limited to exemplary embodiments of the present disclosure set forth herein.

Accordingly, while the present disclosure is capable of various modifications and alternative forms, specific exemplary embodiments thereof are shown by way of example in the drawings and will herein be described in detail. It should be understood, however, that there is no intent to limit the present disclosure to the particular forms disclosed, but on the contrary, the present disclosure is to cover all modifications, equivalents, and alternatives falling within the spirit and scope of the present disclosure. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between,” “adjacent” versus “directly adjacent,” etc.).

The terminology used herein is for the purpose of describing particular exemplary embodiments only and is not intended to be limiting of the present disclosure. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, exemplary embodiments of the present disclosure will be described in greater detail with reference to the accompanying drawings. In order to facilitate general understanding in describing the present disclosure, the same components in the drawings are denoted with the same reference signs, and repeated description thereof will be omitted.

It is known that a pulse transit time (PTT) and heartbeat shape are related to blood pressure. A device such as an electrocardiogram (ECG) device or a photoplethysmogram (PPG) device may obtain relatively accurate information because the device is brought into direct contact with a person's skin and extracts heartbeat-related information. However, in a non-contact measurement method such as a method of measuring blood pressure using a radar, it is difficult to extract information about blood pressure. Unlike the method of measuring blood pressure using an ECG or PPG, in the method of measuring blood pressure using a radar, the blood pressure is measured based on minute movements of the heart and/or wrist blood vessels, rather than directly measuring heartbeat signals or the movement of blood. In particular, when a PTT is measured using a radar, the two signals reflected and received from the heart and wrist blood vessels should be accurately synchronized. This is because the PTT is accurately measured only when measured at the same starting point. In order to measure the PTT at the same starting point using a radar, a method of measuring blood pressure using a synchronized radar may be used.

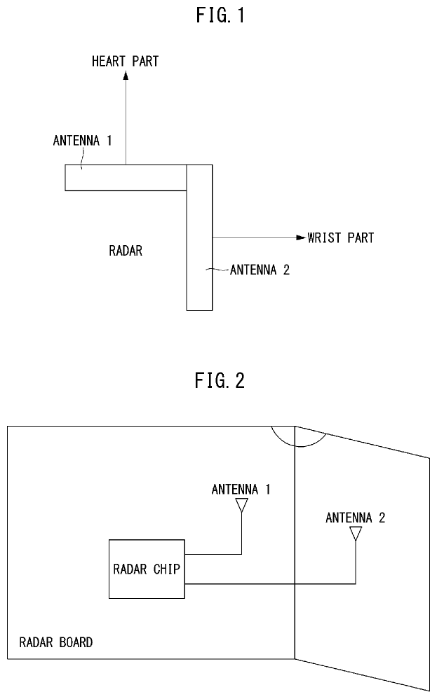

FIG. 1 is a schematic diagram illustrating a method of measuring blood pressure using a synchronized radar according to an embodiment.

Referring to FIG. 1, an antenna 1 of the radar transmits a radar signal to a heart part and receives a radar signal reflected by the heart part. The movement of the heart may be measured using the radar signal reflected by the heart part.

An antenna 2 of the radar transmits a radar signal to a wrist part and receives a radar signal reflected by the wrist part. The movement of the wrist part may be measured using the radar signal reflected by the wrist part.

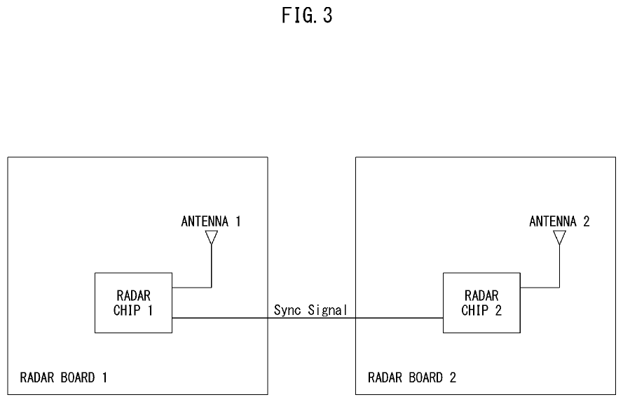

In this case, the synchronized radar may be implemented in the same manner as in FIGS. 2 and 3.

FIGS. 2 and 3 are diagrams each illustrating an example of a configuration of a board of the synchronized radar.

As illustrated in FIG. 2, a radar chip is mounted on a radar board, and an antenna 1 and an antenna 2 are connected to the radar chip. In order to synchronize the antenna 1 and the antenna 2, the radar board may be bent so that the antenna 1 and the antenna 2 face a corresponding body part according to a position of the body part to be measured.

Unlike the above case, as illustrated in FIG. 3, an antenna 1 and an antenna 2 are mounted on separate radar boards, and a synchronization signal may be used for synchronization between the two radar boards.

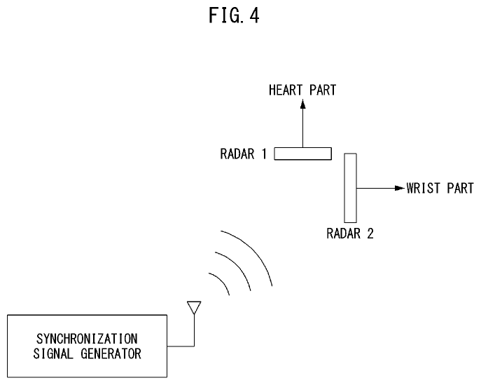

When the synchronized radar configured as illustrated in FIGS. 2 and 3 is used, the antenna 1 and the antenna 2 may be directly synchronized, but there may be a restriction on determining the positions of the antennas. In this case, an unsynchronized radar may be used as illustrated in FIG. 4.

FIG. 4 is a schematic diagram illustrating a method of measuring blood pressure using two unsynchronized radars.

Referring to FIG. 4, when two unsynchronized radars are used, an external synchronization signal generator may be used for synchronization between signals obtained from a radar 1 and a radar 2. The radar 1 and the radar 2 simultaneously receive a synchronization signal transmitted from the synchronization signal generator. The signals obtained from the radar 1 and the radar 2 may be synchronized using the synchronization signal.

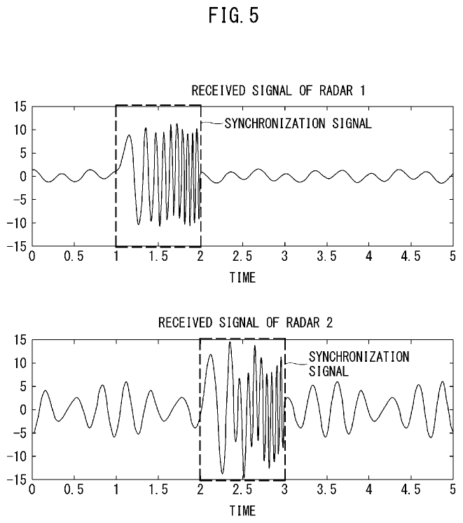

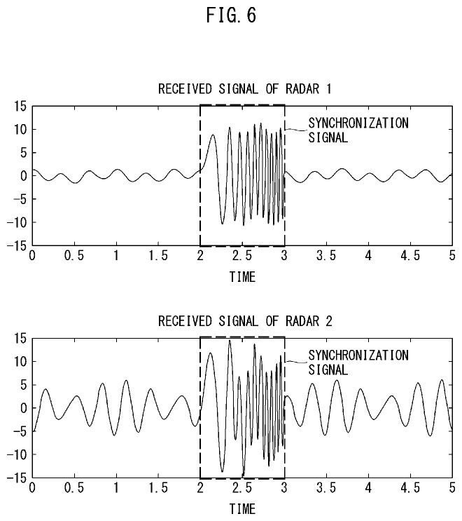

FIG. 5 is a set of diagrams showing examples of received signals of two unsynchronized radars, and FIG. 6 is a set of diagrams showing examples of received signals of two synchronized radars.

Referring to FIGS. 5 and 6, a radar 1 and a radar 2 receive signals obtained by adding a biosignal to a synchronization signal output from a synchronization signal generator.

Generally, since the magnitude of a synchronization signal is greater than that of a biosignal, synchronization between signals received from the radar 1 and the radar 2 may be achieved using information about the magnitude of the synchronization signal, as shown in FIG. 6. Alternatively, a chirp signal may be transmitted as a synchronization signal so that a position of the synchronization signal may be accurately tracked using pulse compression. Alternatively, a synchronization signal may be generated using a code as in a direct-sequence spread spectrum (DSSS) method, and synchronization between signals may be achieved using the synchronization signal.

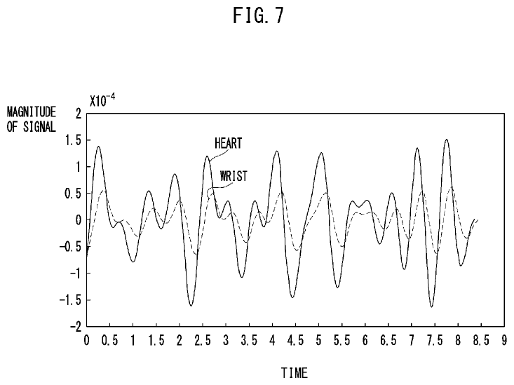

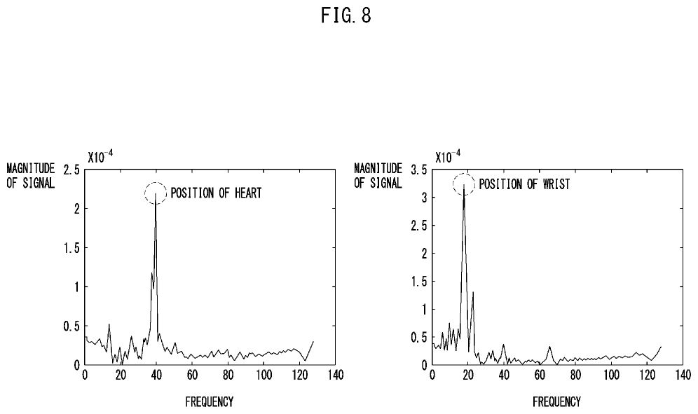

FIG. 7 is a diagram showing examples of radar signals in a synchronized time domain, and FIG. 8 is a set of diagrams showing examples of radar signals in a synchronized frequency domain.

Referring to FIGS. 7 and 8, a radar 1 and a radar 2 receive radar signals reflected by a heart part and a wrist part, respectively.

Since the two radar signals may contain a mixture of a breathing-related biosignal and noise, signals not related to heartbeats are removed through filtering. After the filtering process is performed, a PTT may be obtained using a time difference between two signals in a time domain, as shown in FIG. 7. Alternatively, as shown in FIG. 8, a PTT may be obtained using a phase difference between two signals in a frequency domain.

In order to measure blood pressure using information about the PTT of the radar signal, a relational expression between PTT and blood pressure should first be derived.

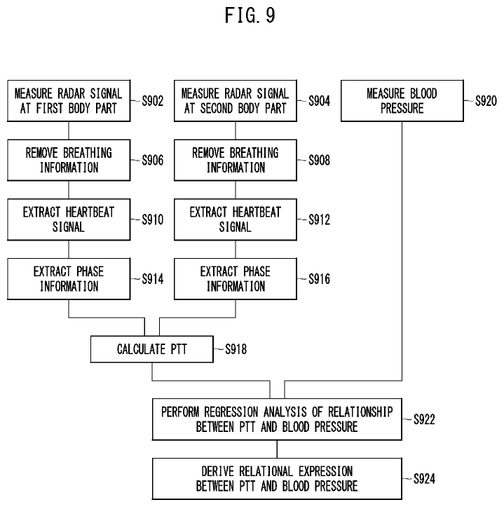

FIG. 9 is a diagram illustrating a process of deriving a relational expression between PTT and blood pressure.

Referring to FIG. 9, a blood pressure measurement apparatus measures a radar signal at each of first and second body parts (S902 and S904). The first body part may be a heart part, and the second body part may be a wrist part. The radar signal measurement may include a process of transmitting radar signals and receiving radar signals reflected by the corresponding body parts.

Only a heartbeat signal is required to obtain a PTT. Therefore, the blood pressure measurement apparatus removes breathing information or other noise mixed in each radar signal through filtering (S906 and S908) and extracts a heartbeat signal corresponding to heartbeat information (S910 and S912). Thereafter, after frequency conversion is performed on the heartbeat signal, a frequency component corresponding to the corresponding heartbeat signal is selected, and phase information of the corresponding frequency component is extracted (S914 and S916).

The blood pressure measurement apparatus calculates the PTT using the phase information extracted from the radar signals measured at the first and second signal parts (S918).

Meanwhile, blood pressure and the radar signals are simultaneously measured (S920). In this case, the blood pressure may be measured using, for example, a cuff.

The blood pressure measurement apparatus performs regression analysis using the PTT and information about the measured blood pressure (S922), and derives a relational expression between PTT and blood pressure using a result of the regression analysis (S924).

The derivation of such a relational expression may be performed by the blood pressure measurement apparatus or may be performed by an apparatus separate from the blood pressure measurement apparatus.

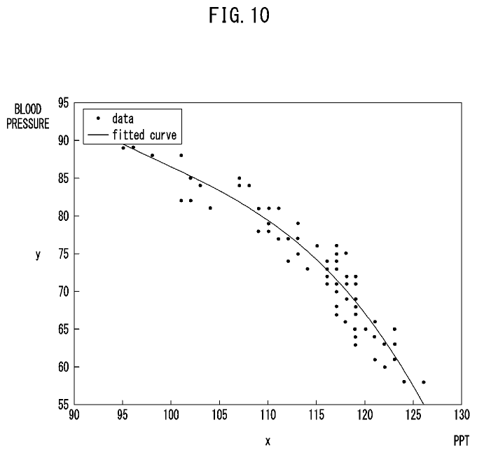

FIG. 10 is a diagram showing an example of the relational expression between PTT and blood pressure derived by the method illustrated in FIG. 9.

As shown in FIG. 10, when the relational expression between PTT and blood pressure is derived, the blood pressure measurement apparatus may extract information about blood pressure using the relational expression between PTT and blood pressure.

That is, when the blood pressure measurement apparatus is intended to measure blood pressure, the blood pressure measurement apparatus calculates the PTT using the phase information extracted from the radar signal measured at each body part by performing the processes (S902 to S916) described in FIG. 9. Next, the blood pressure measurement apparatus may calculate the blood pressure from the PTT using the relational expression between PTT and blood pressure.

As described above, the blood pressure measurement apparatus may extract the information about blood pressure using only the radar signal using the relational expression between PTT and blood pressure.

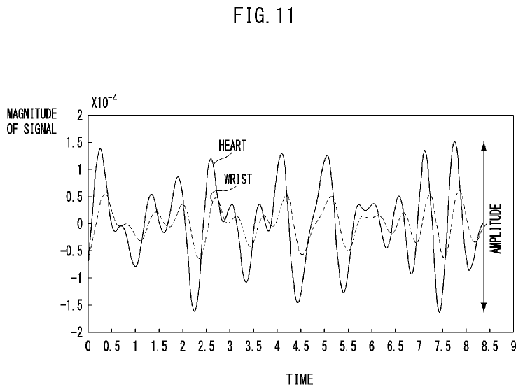

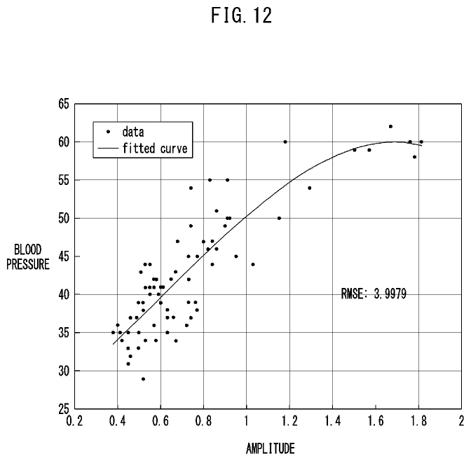

FIG. 11 is a diagram showing examples of amplitude information of radar signals measured at a heart part and a wrist part, and FIG. 12 is a diagram showing a relationship between the amplitude of a radar signal and blood pressure.

Referring to FIGS. 11 and 12, the amplitude of a radar signal measured at each body part is also related to blood pressure. Therefore, information about blood pressure may be extracted using information about the amplitude. In this case, using one radar, information about the amplitude of a radar signal measured at one body part may be used or a difference between pieces of information about the amplitude of radar signals measured at two or more body parts may be used.

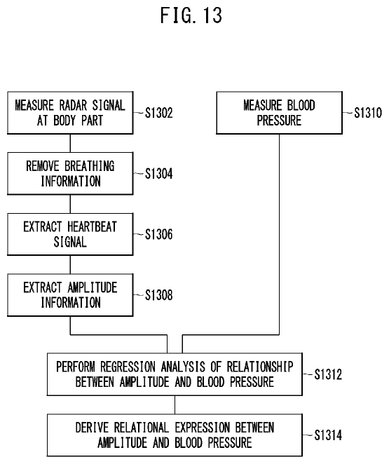

FIG. 13 is a diagram illustrating a process of deriving a relational expression between information about the amplitude of one radar signal and blood pressure.

Referring to FIG. 13, the blood pressure measurement apparatus measures a radar signal at a body part (S1302). The radar signal measurement may include a process of transmitting a radar signal and receiving a radar signal reflected by the corresponding body part.

The blood pressure measurement apparatus removes breathing information or other noise mixed in the radar signal through filtering (S1304) and extracts a heartbeat signal corresponding to heartbeat information (S1306). Thereafter, information about the amplitude of the heartbeat signal is extracted (S1308).

Meanwhile, blood pressure and the radar signal are simultaneously measured (S1310).

The blood pressure measurement apparatus performs regression analysis using the extracted information about the amplitude and information about the measured blood pressure (S1312), and derives a relational expression between amplitude and blood pressure using a result of the regression analysis (S1314).

In this way, when the relational expression between amplitude and blood pressure is derived, the blood pressure measurement apparatus may extract information about blood pressure using the relational expression between amplitude and blood pressure.

That is, when the blood pressure measurement apparatus is intended to measure blood pressure, the blood pressure measurement apparatus calculates the information about the amplitude from the radar signal measured at the body part by performing the processes (S1302 to S1308) described in FIG. 13. Next, the blood pressure measurement apparatus may calculate the blood pressure from the information about the amplitude calculated using the relational expression between amplitude and blood pressure.

As described above, the blood pressure measurement apparatus may extract the information about blood pressure using only the radar signal using the relational expression between amplitude and blood pressure.

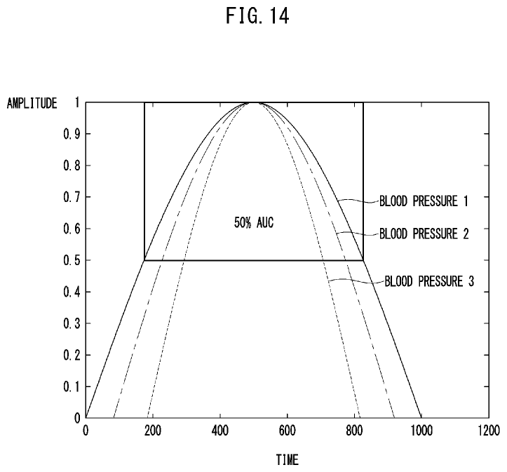

FIG. 14 is a diagram showing a relationship between a shape of a radar signal and blood pressure.

Referring to FIG. 14, in general, the more severe the curvature at a peak point of a signal, the higher the blood pressure, and the gentler the curvature at the peak point, the lower the blood pressure. That is, in FIG. 14, blood pressure 3 represents the highest blood pressure, and blood pressure 1 represents the lowest blood pressure.

As described above, information about the shape of the radar signal is related to blood pressure. As the magnitude of curvature at a peak point of the radar signal increases, an area under the curve (AUC) representing an area under the shape drawn by a curve decreases. A value of the AUC of the radar signal has information related to the shape of the curve, which means that it is related to blood pressure. FIG. 14 shows examples of an AUC obtained at a point of an amplitude of 0.5 after normalizing the magnitude of the signal to 1. Therefore, blood pressure may be measured using the information about the shape of the radar signal.

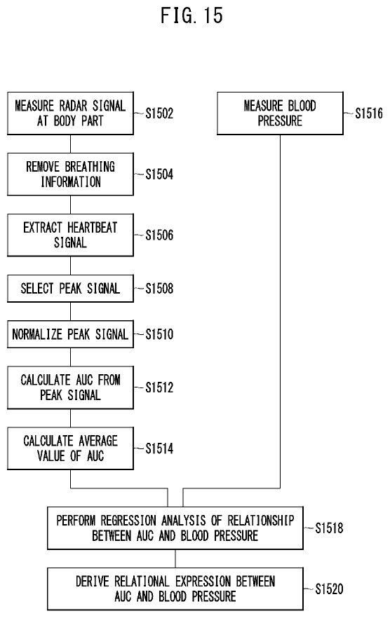

FIG. 15 is a flowchart illustrating a method of deriving a relational expression between AUC measured from a radar signal and blood pressure.

Referring to FIG. 15, the blood pressure measurement apparatus measures a radar signal at a body part (S1502). The radar signal measurement may include a process of transmitting a radar signal and receiving a radar signal reflected by the corresponding body part.

The blood pressure measurement apparatus removes breathing information or other noise mixed in the radar signal through filtering (S1504) and extracts a heartbeat signal corresponding to heartbeat information (S1506).

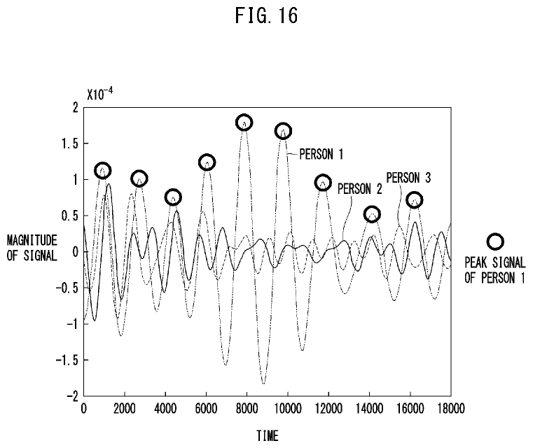

FIG. 16 is a diagram showing an example of a heartbeat signal extracted from a radar signal.

Referring to FIG. 16, the heartbeat signal appears as a signal having several peaks in a time domain.

Referring to FIG. 15 again, the blood pressure measurement apparatus selects, from among peak signals present in the heartbeat signal, several peak signals for which AUCs are to be measured (S1508).

The blood pressure measurement apparatus normalizes the magnitudes of the selected peak signals (S1510) and calculates the AUCs on the basis of the normalized magnitudes of the peak signals (S1512).

The blood pressure measurement apparatus calculates an average value of the AUCs measured from the several peak signals (S1514). In this case, different weights may be given according to the magnitudes of the peak signals.

Meanwhile, blood pressure and the radar signals are simultaneously measured (S1516).

The blood pressure measurement apparatus performs regression analysis using the average value of the AUCs and information about the measured blood pressure (S1518), and derives a relational expression between AUC and blood pressure using a result of the regression analysis (S1520).

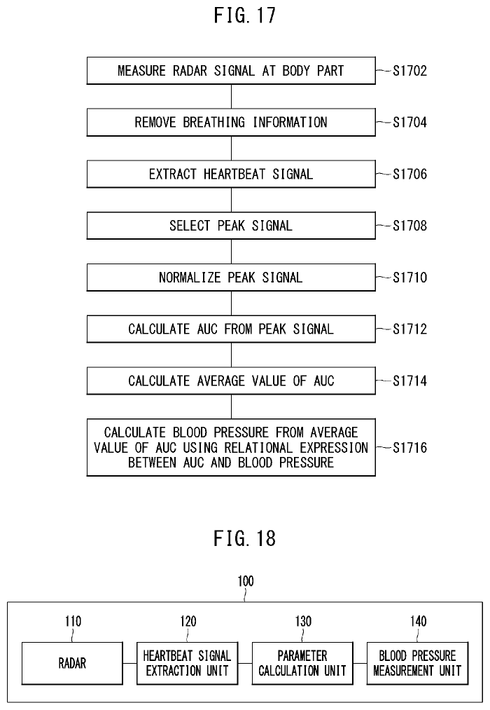

FIG. 17 is a flowchart illustrating a method of measuring blood pressure using an AUC measured from a radar signal according to an embodiment.

Referring to FIG. 17, when the blood pressure measurement apparatus is intended to measure blood pressure, the blood pressure measurement apparatus measures a radar signal at a body part (S1702). The radar signal measurement may include a process of transmitting a radar signal and receiving a radar signal reflected by the corresponding body part.

The blood pressure measurement apparatus removes breathing information or other noise from the measured radar signal through filtering (S1704) and extracts a heartbeat signal corresponding to heartbeat information (S1706).

The blood pressure measurement apparatus selects several peak signals, for which AUCs are to be measured, from the heartbeat signal (S1708).

The blood pressure measurement apparatus normalizes the magnitudes of the selected peak signals (S1710) and calculates the AUCs on the basis of the normalized magnitudes of the peak signals (S1712).

The blood pressure measurement apparatus calculates an average value of the AUCs measured from the several peak signals (S1714).

The blood pressure measurement apparatus calculates blood pressure corresponding to the average value of the AUCs using a relational expression between AUC and blood pressure (S1716).

As described above, the blood pressure measurement apparatus may extract the information about blood pressure using only the radar signal using the relational expression between AUC and blood pressure.

As described above, the blood pressure measurement apparatus may calculate the PTT, the amplitude, the AUC, or the like from the radar signal, and measure the blood pressure using the calculated PTT, amplitude, AUC, or the like.

Meanwhile, the blood pressure measurement apparatus may measure blood pressure using the information about the PTT, amplitude, and AUC alone, or may measure blood pressure using a combination of two or more of the PTT, the amplitude, and the AUC to increase the accuracy of blood pressure measurement. That is, one parameter representing the combination of two or more of the PTT, the amplitude, and the AUC may be defined, and blood pressure may be calculated by calculating a value of the parameter.

For example, a parameter X representing a combination of the PTT, the amplitude, and the AUC may be defined and calculated as shown in Equation 1, and blood pressure may be calculated using the calculated value of the parameter X.

X=a*PTT+b*amplitude+c*AUC [Equation 1]

Here, a, b, and c denote weights applied to the PTT, the amplitude, and the AUC, respectively, and different weight values may be applied to the PTT, the amplitude, and the AUC.

As described above, in the method of calculating blood pressure using the calculated value of the parameter X, a relational expression between the parameter X representing the combination of the PTT, the amplitude, and the AUC and blood pressure may be pre-derived, and blood pressure may be calculated from the parameter X calculated using the pre-derived relational expression between the parameter X and blood pressure.

For example, the parameter X may be defined as a combination of the AUC and the amplitude or may be defined as a combination of the AUC and the PTT.

FIG. 18 is a diagram illustrating a blood pressure measurement apparatus using a radar according to an embodiment.

Referring to FIG. 18, a blood pressure measurement apparatus 100 using a radar includes at least one radar 110, a heartbeat signal extraction unit 120, a parameter calculation unit 130, and a blood pressure measurement unit 140.

The radar 110 transmits a radar signal to a body part and receives a radar signal reflected by a body part. The body part may be a heart part or a wrist part. When two radars are used as illustrated in FIG. 4, a radar 1 may transmit a radar signal to a heart part, and a radar 2 may transmit a radar signal to a wrist part.

The heartbeat signal extraction unit 120 removes breathing information or other noise from the received radar signal and extracts a heartbeat signal corresponding to heartbeat information.

The parameter calculation unit 130 calculates a parameter to be used for measuring blood pressure from a heartbeat signal. The parameter calculation unit 130 may calculate a PTT from heartbeat signals of the heart part and the wrist part, may calculate information about the amplitude of the heartbeat signal from the heartbeat signal of the heart part and/or the wrist part, or may calculate an average value of AUCs as described in FIG. 18.

The blood pressure measurement unit 140 measures blood pressure using a relational expression between the parameter calculated by the parameter calculation unit 130 and blood pressure. The blood pressure measurement unit 140 may calculate blood pressure from the average value of the AUCs using a relational expression between AUC and blood pressure. The blood pressure measurement unit 140 may calculate the blood pressure from the PTT calculated using a relational expression between PTT and blood pressure. The blood pressure measurement unit 140 may calculate blood pressure from the information about the amplitude of the heartbeat signal calculated using a relational expression between amplitude and blood pressure. The blood pressure measurement unit 140 may measure blood pressure through a combination of the pieces of information about the PTT, the amplitude, and the AUC to increase the accuracy of the blood pressure measurement.

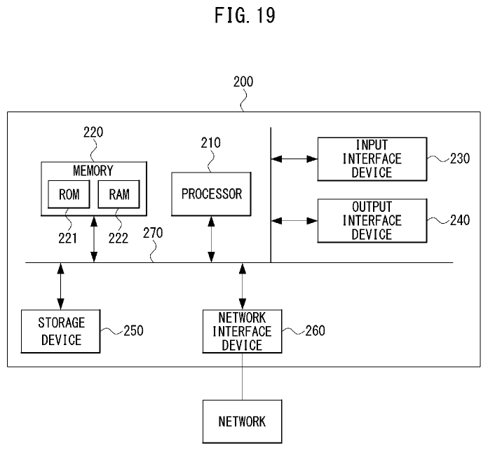

FIG. 19 is a diagram illustrating a blood pressure measurement apparatus using a radar according to another embodiment.

Referring to FIG. 19, a blood pressure measurement apparatus 200 using a radar may represent a computing device in which the blood pressure measurement method using the radar described above is implemented.

The blood pressure measurement apparatus 200 using a radar may include at least one of a processor 210, a memory 220, an input interface device 230, an output interface device 240, a storage device 250, and a network interface device 260. The respective components may be connected to each other through a common bus 270 to communicate with each other. Further, the respective components may be connected to each other through individual interfaces or individual buses with the processor 210 positioned therebetween, instead of the common bus 270.

The processor 210 may be implemented with various types of devices such as an application processor (AP), a central processing unit (CPU), a graphics processing unit (GPU), and the like, and may be a semiconductor device that executes instructions stored in the memory 220 or storage device 250. The processor 210 may execute program instructions stored in at least one of the memory 220 and the storage device 250. The processor 210 may store program instructions for implementing at least some functions of the heartbeat signal extraction unit 120, the parameter calculation unit 130, and the blood pressure measurement unit 140 described in FIG. 17 in the memory 220, and control the operations described with reference to FIGS. 1 to 18 to be performed.

The memory 220 and the storage device 250 may include various types of volatile or non-volatile storage media. For example, the memory 220 may include a read-only memory (ROM) 221 and a random-access memory (RAM) 222. The memory 220 may be positioned inside or outside the processor 210, and the memory 220 may be connected to the processor 210 through various known means.

The input interface device 230 is configured to provide data to the processor 210. For example, the input interface device 230 may provide the received radar signal to the processor 210.

The output interface device 240 is configured to output data received from the processor 210. For example, the output interface device 240 may output information about the measured blood pressure.

The network interface device 260 may transmit or receive a signal to or from an external device through a wired network or a wireless network.

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

What is claimed is:

1. A blood pressure measurement method using at least one radar in a blood pressure measurement apparatus, comprising:
measuring a radar signal for at least one body part;
extracting a heartbeat signal from the radar signal;
selecting one or more peak signals from the heartbeat signal;
calculating an area under a curve (AUC) of each of the selected one or more peak signals; and
calculating blood pressure from the calculated AUC on the basis of a derived relational expression between AUC and blood pressure.

2. The blood pressure measurement method of claim 1, wherein the calculating of the AUC includes calculating an average value of the calculated AUCs from the selected one or more peak signals.

3. The blood pressure measurement method of claim 1, wherein the calculating of the AUC includes normalizing a magnitude of the selected peak signal before the AUC is calculated.

4. The blood pressure measurement method of claim 1, wherein the extracting of the heartbeat signal includes removing a signal related to breathing information and a noise signal from the radar signal.

5. A blood pressure measurement apparatus using at least one radar, comprising:
a heartbeat signal extraction unit configured to extract a heartbeat signal from a radar signal reflected by at least one body part through the at least one radar; and
a blood pressure measurement unit configured to calculate a parameter to be used for measuring blood pressure from the heartbeat signal and calculate the blood pressure from the parameter,
wherein the blood pressure measurement unit selects one or more peak signals from the heartbeat signal, calculates an area under curve (AUC) of each of the selected one or more peak signals, and calculates the blood pressure using the calculated AUC corresponding to the parameter.

6. The blood pressure measurement apparatus of claim 5, wherein the blood pressure measurement unit calculates the blood pressure from the calculated AUC on the basis of a derived relational expression between AUC and blood pressure.

7. The blood pressure measurement apparatus of claim 5, wherein the blood pressure measurement unit extracts amplitude information of the heartbeat signal and calculates the parameter by combining the calculated AUC and the extracted amplitude information.

8. The blood pressure measurement apparatus of claim 5, wherein the blood pressure measurement unit extracts phase information of a frequency component from a radar signal reflected by each of two body parts, calculates a pulse transit time (PTT) using the extracted phase information, and calculates the parameter by combining the calculated AUC and the calculated PTT.

9. The blood pressure measurement apparatus of claim 5, wherein the heartbeat signal extraction unit removes a signal related to breathing information and a noise signal from the radar signal.

10. The blood pressure measurement apparatus of claim 5, wherein the blood pressure measurement unit extracts amplitude information of the heartbeat signal, calculates a PTT from a radar signal reflected by each of two body parts, and calculates the parameter by combining the calculated AUC, the extracted amplitude information, and the calculated PTT.
